(19) United States
(12) Patent Application Publication
(10) Pub. No.: US 2016/0208389 A1
(43) Pub. Date: Jul. 21, 2016

(54) Method for Improving the Biocompatibility of a Surface

(71) Applicant: GILUPI GmbH, Postdam (DE)

(72) Inventors: Andreas Bollmann, Berlin (DE); Klaus Lücke, Werder (DE); Fritz Scholz, Greifswald (DE); Katja Vahl, Greifswald (DE); Robert Smail, Berlin (DE); Ulrich Hasse, Rankwitz (DE); Heike Kahlert, Guest (DE)

(21) Appl. No.: 14/914,916

(22) PCT Filed: Aug. 27, 2014
(86) PCT No.: PCT/EP2014/068160

§ 371 Date: Feb. 26, 2016

(30) Foreign Application Priority Data
Aug. 27, 2013 (DE) 10 2013 217 085.8

(51) Int. Cl. C23C 22/05; A61L 31/14; A61L 31/02

(52) U.S. Cl. CPC C23C 22/05; A61L 31/022; A61L 31/14; A61L 2400/18

(57) ABSTRACT

The present invention relates to a method for improving the biocompatibility of a surface, in particular a solid body surface, as well as a device, for example an implant, sensor or cell culture vessel, which is brought in contact with biological systems with a biocompatible surface. To improve the biocompatibility of surfaces, in particular in relation to cell cultures and tissues, in a simple way and for a plurality of different surfaces, the surface is meant to be brought in contact with reactive radicals according to the invention. The device according to the invention has a biocompatible surface that has been treated pursuant to the method according to the invention.

The present invention relates to a method for improving the biocompatibility of a surface, in particular a solid body surface.

The invention further relates to a device, for example an implant, a sensor or a cell culture vessel that is brought in contact with biological systems, with a biocompatible surface.

Materials that come in contact with biological systems must have a high biocompatibility, i.e. (I) the materials must not have a damaging effect on the biological system and (II) the biological environment must not cause any material changes such as corrosion, biodegradation, etc.

To increase the biocompatibility of materials, there is a variety of mechanical, chemical and physical methods for the modification of surfaces. Through mechanical modification (e.g. through polishing or grinding), defined surface topographies or roughness levels should be achieved, impurities of the surface should be removed and the adhesion properties for subsequent bonding processes of molecules should be improved (I. Milinković, R. Rudolf, K. T. Raić, Z. Aleksić, V. Lazić, A. Todorović, D. Stamenković, Materiali in tehnologije/Materials and Technology 46 (2012) 251-256).

Surfaces can be chemically modified through direct reactions with specific reagents, through covalent bonding of molecules on the surface, through plasma-based techniques such as plasma-supported etching, deposition or polymerization as well as plasma-immersion ion implantation (P. K. Chu, J. Y. Chen, L. P. Wang, N. Huang, Mater. Sci. Eng., R 36 (2002) 143-206).

Liu, Chu and Ding provide an overview of different possibilities of surface modification of titanium and titanium alloys for biomedical applications (X. Liu, P. K. Chu, C. Ding, Mater. Sci. Eng., R 47 (2004) 49-121). Titanium surfaces can be treated chemically with acids or caustic solutions. In addition, the chemical modifications include sol-gel coatings, anodic oxidations, chemical gas phase depositions as well as biochemical modifications. In a physical way, titanium surfaces can be modified through thermal spraying (e.g. flame spraying or plasma spraying), through physical gas phase deposition or through ion implantation and ion deposition. In electrochemical terms, the biocompatibility of titanium surfaces can be increased by means of anodic oxidation and through electrophoretic or cathodic deposition of hydroxylapatite (K.-H. Kim, N. Ramaswamy, Dent. Mater. J. 28 (2009) 20-36).

Further, different physical and chemical methods are described for the modification of polymer surfaces (F. Abbasi, H. Mirzadeh, A.-A. Katbab, Polym. Int. 50 (2001) 1279-1287). The most common physical methods for surface modification of silicone polymers are plasma and laser treatments as well as corona discharges. The surfaces of silicone polymers can be modified chemically through etching, oxidation, hydrolysis, functionalization as well as “surface grafting”.

For all mentioned methods, the properties of surfaces can be changed in order to increase the biocompatibility in various ways.

The object of the present invention is to provide a method for the treatment of surfaces that allows for an improvement of the biocompatibility of the surface, in particular with regard to cell cultures and tissues, in a simple way and that can be used for a plurality of different surfaces, in particular for solid body surfaces.

The abovementioned object is achieved according to the invention in that the surface is treated with at least one species of reactive radicals. The initially mentioned device solves the problem due to the condition that its biocompatible surface has been treated with a method of the present invention.

According to the invention, it became apparent by surprise that a surface treatment with at least one species of reactive radicals detoxifies the surface and improves the biocompatibility of the surface in this way. Contrary to the existing methods, the surface is detoxified by the reactive radicals, which increases their biocompatibility in relation to biological systems without adding for example additional layers to the surface.

A further advantage is that the radicals can be generated in very different ways and that the method can therefore be adapted to diverse material requirements. If the goal is to increase the biocompatibility for example of heat-sensitive surfaces, the radicals can for example be produced at ambient temperature by means of the Fenton reaction. If the surface is to be treated with minimal use of chemicals, photolysis or radiolysis can for example be used for the creation of radicals.

A “reactive radical” is an atom or molecule with at least one unpaired electron that is reactive. Reactive radicals usually react very quickly, often within less than a second. At least one species of reactive radicals (“Wenigstens eine Spezies von reaktiven Radikalen”) comprises embodiments in which the surface is treated only with a single type of radicals (radical atoms, radical ions, radical molecules or radial molecule ions) as well as those in which different types of radicals come in contact with the surface. An improvement of the biocompatibility (“Verbesserung der Biokompatibilität”) in the sense of the present invention becomes apparent through a detoxification of the surface, i.e. the treated surface according to the invention is less cytotoxic, i.e. less cell- and/or tissue-damaging compared to an untreated surface that has not been brought in contact with reactive radicals. The improved biocompatibility can be determined by means of a cytotoxicity test in which the untreated surface and, in one occasion, the surface treated with reactive radicals is brought in contact with a cell culture and in which the cell vitality is subsequently determined in the solution. By means of the method according to the invention, the cell vitality can be increased by at least 10%, preferably by at least 25% and particularly preferably by 50-100%.

The solution according to the invention can be further improved through different embodiments that are each advantageous in isolation and that can be combined arbitrarily with each other in any way. These embodiments and the related advantages will be addressed in the following.

According to an embodiment of the method, the reactive radicals can deactivate active centers of the surface that trigger biological reactions and that have a cell- and/or tissue-damaging effect. Hence, the active centers on the surface that trigger cytotoxic reactions are deactivated systematically and specifically through the treatment with reactive radicals. This is surprising and unexpected because one would expect reactive radicals to trigger chemical reactions on the surface that generate active centers and therefore have a cytotoxic effect. An active center that triggers cytotoxic reactions is an atom or a substance on the cell surface that has a cell- and/or tissue-damaging effect. By means of reactive radicals, these active centers can be deactivated systematically and specifically, for example by transforming them into non-cytotoxic substances or by extracting them from the cell surface, for instance through reactive splitting/reactive breakdown.

According to a further embodiment, the reactive radicals can comprise at least one species of oxygen radicals, nitrogen radicals, carbon radicals, sulfur radicals and/or a species of halogen radicals. Reactive oxygen radicals include all radicals in which the at least single unpaired electron sits on an oxygen atom. Examples of oxygen radicals are hyperoxide anions, hydroxyl radicals, hydroperoxyl radicals, peroxyl radicals or alcoxyl radicals. Examples for nitrogen radicals are nitrogen monoxide or tri-nitrogen. Carbon radicals comprise for example triplet carben and alkyl radicals, and sulfur radicals include for example thiyl radicals. Halogen radicals comprise, inter alia, chlorine radicals and bromine radicals.

According to a further embodiment, reactive radicals can be created by means of breaking down a radical starter. A radical starter is a molecule that can be transformed into at least one reactive radical. For example, the chlorine-chlorine bond in molecular chlorine (Cl₂) or the bromine-bromine bond in molecular bromine (Br₂) can be split through the impact of light whereby the molecular radical starters are transformed into reactive radicals.

According to an embodiment, the surface can be brought in contact with the radical starter that is usually stable in contrast to reactive radicals, and the radical starter can subsequently be transformed in situ into the reactive radical. This way, it can be ensured that the overall surface will be treated evenly.

The radical starter can be transformed into the reactive radical by means of photolysis, radiolysis, thermolysis, by means of plasma and/or through a chemical, for example electrochemical, and/or a biochemical, for example an enzymatic, reaction. The radical creation can therefore occur in different ways and in adaptation to the properties of the surface to be treated, for example non-thermally by means of light, for example UV radiation, or using x-rays or other ionizing radiation. A chemical transformation, for example in form of a chemical or electrochemical Fenton reaction, in which hydrogen peroxide is decomposed through the reaction with Fe(II) ions or also with other transitional metal ions such as Cu(II), Ti(III), Cr(II) or Co(II) in an acidic medium while forming the highly reactive hydroxyl radical, is also possible at ambient temperature.

According to a further embodiment of the method according to the invention, the reactive radical can be a hydroxyl radical. Hydroxyl radicals can be created in a simple way of harmless substances such as water. Hydroxyl radicals can in particular be formed:
a) in a Fenton reaction;
b) through photolysis of a peroxide;
c) through radiolysis of water or another oxygen compound that can be radiolyzed into hydroxyl radicals; or
d) through a plasma reaction of an oxygen compound that can be transformed into hydroxyl radicals by means of plasma treatment, preferably of water or a peroxide.

The surface whose biocompatibility is improved by means of the method according to the invention can for example include a precious metal, a precious metal compound and/or alloy or a polymer. Precious metals such as gold are frequently used as electrodes in biosensors and as implant material. Implants and cell culture vessels are often made of polymers that, although they do not cause any material change such as corrosion in a biological environment, have cell- and/or tissue-damaging effects on biological systems and whose biocompatibility can therefore be improved by means of the method according to the invention.

According to a further embodiment, the surface can belong to an implant, a sensor or a cell culture vessel. An advantage is the condition that the implant, the sensor and/or the culture vessel can at first be produced and subsequently treated according to the invention. The method according to the invention is universal, i.e. it can be used for any sort of surface and any surface type because particularly suitable reactive radicals can be used to provide various methods that are adapted to the material requirements for a defined sort of surface and/or a defined surface type.

According to the invention, a device with a biocompatible surface that is brought in contact with biological systems, for example an implant, a sensor or a cell culture vessel that has been treated according to one of the above methods, is further to be provided. The device is characterized by a surface with improved biocompatibility, which can be detected in a simple way due to the condition that, when comparing a surface prior to the treatment with reactive radicals and a surface that has been treated with reactive radicals, the latter shows a much higher cell vitality when it is brought in contact with a cell culture. Another feature of the device according to the invention is the fact that the active centers that trigger biological reactions and that have a cell- and/or tissue-damaging effect are systematically deactivated, i.e. transformed into biologically inactive molecules or, for example in the case of biologically active gold ions, detached from the surface.

In the following, the invention will be explained in greater detail by means of exemplary embodiments with reference to the drawings and specific experiments. The combinations of features shown in the embodiments in an exemplary way can, pursuant to the above explanations, be supplemented by further features in accordance with the properties of the device according to the invention and/or the method according to the invention that are required for a specific case of use. Likewise, and also pursuant to the above explanations, individual features can be omitted for the described embodiments if the effect of this feature is not relevant in a specific case of use.

Identical reference signs are used in the drawings for elements with the same function and/or the same structure.

The figures show:

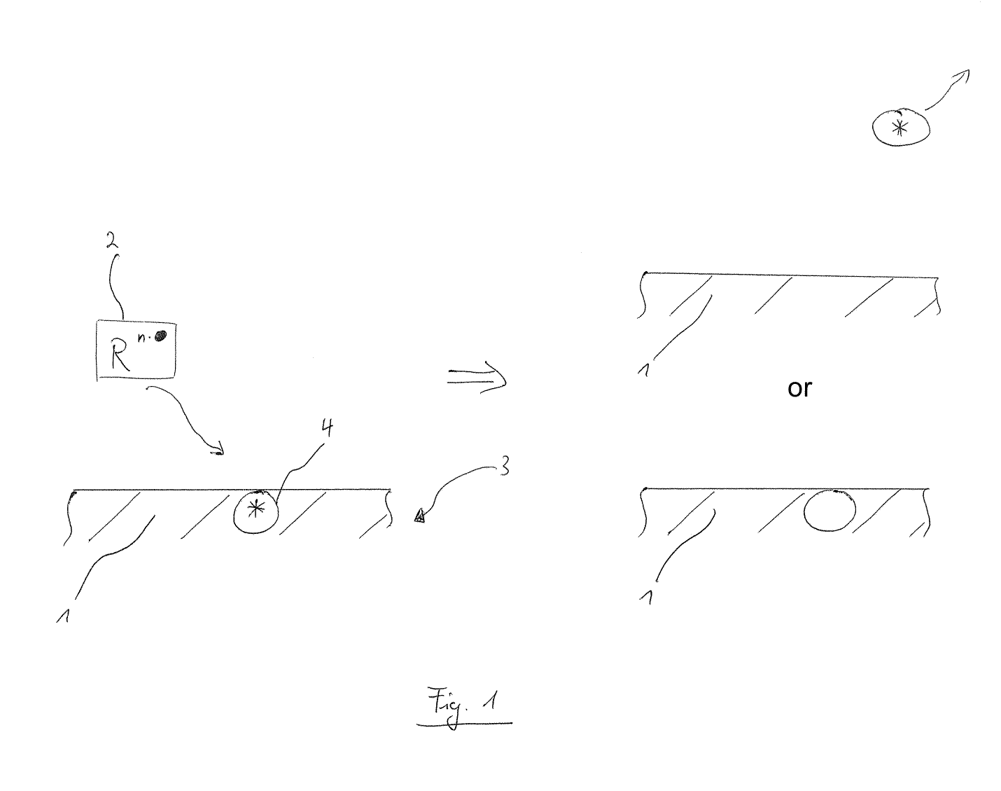

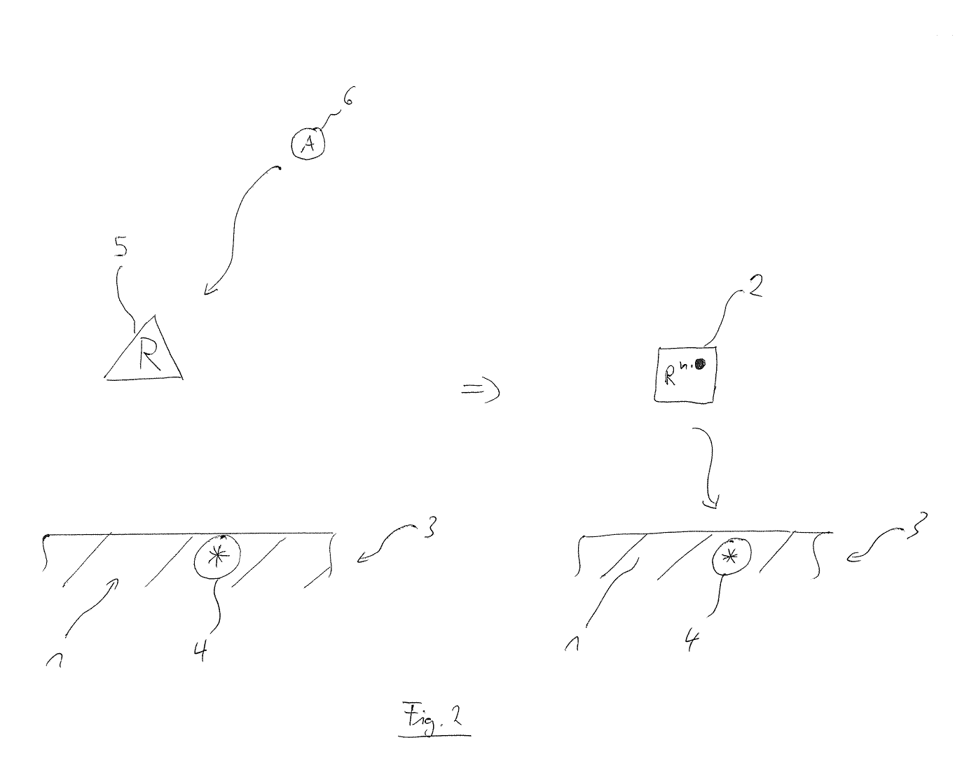

FIG. 1: a schematic display of the method according to the invention for improving the biocompatibility of a surface according to a first embodiment;

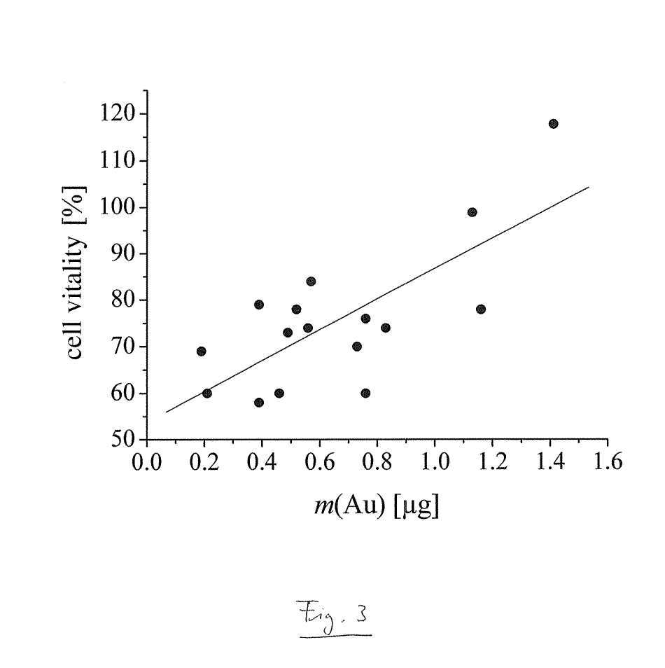

FIG. 2: a schematic display of a method for improving the biocompatibility of a surface according to a second embodiment;

FIG. 3: a graph relating to the cell vitality as a function of the quantity of gold that is detached from a gold surface;

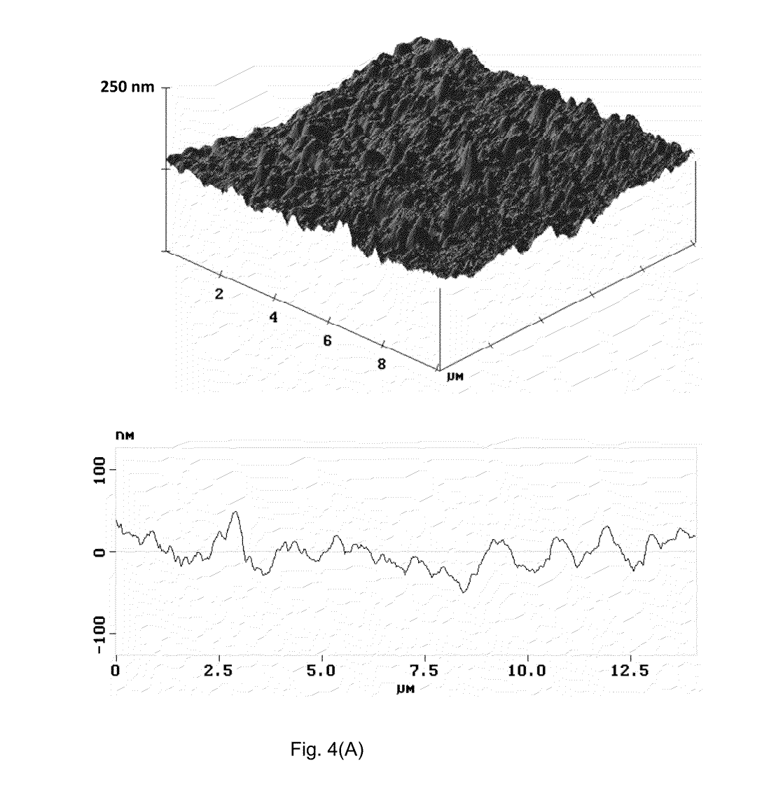

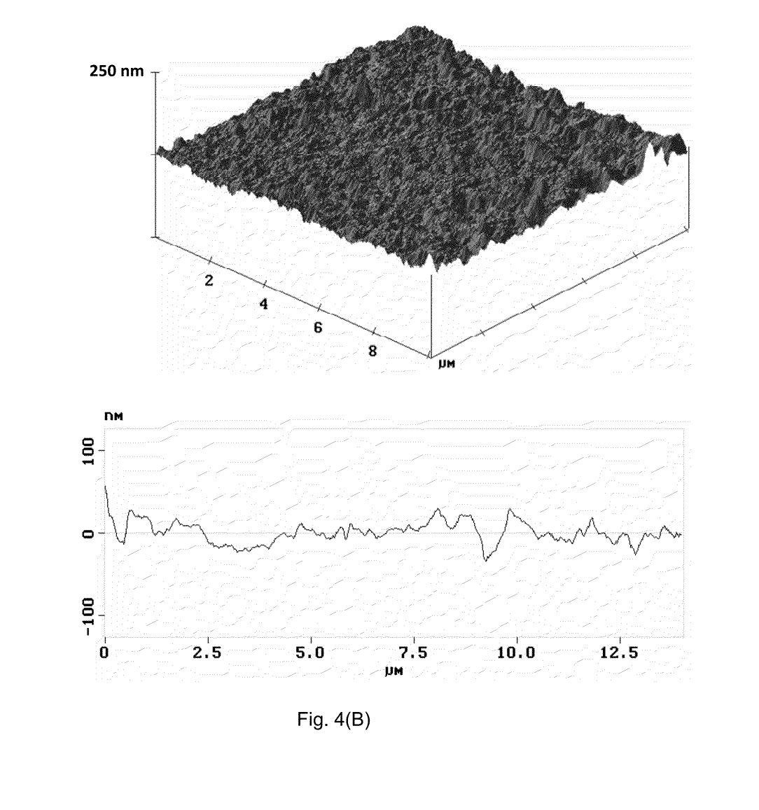

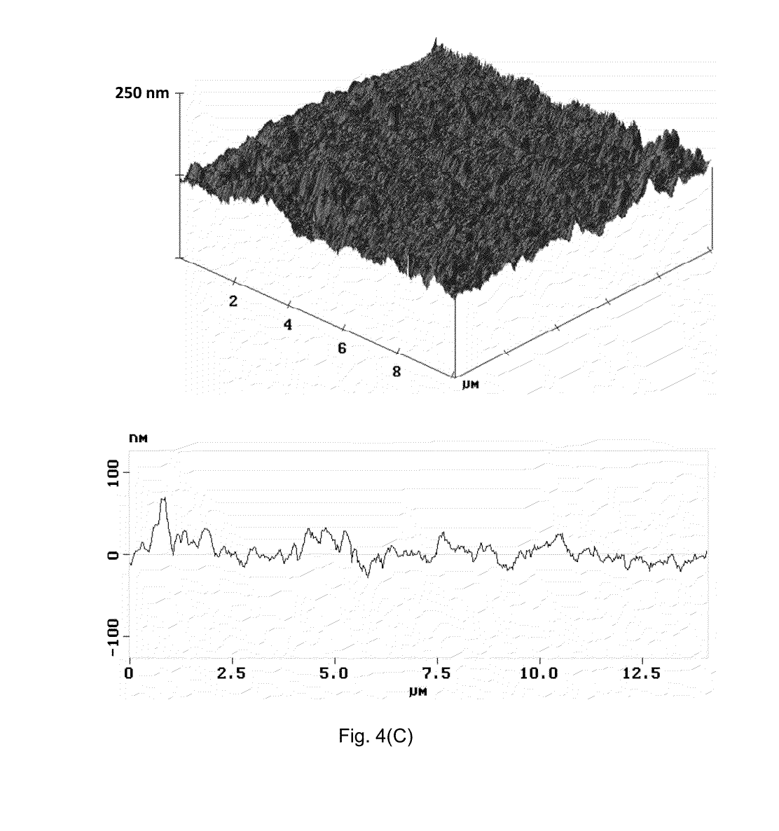

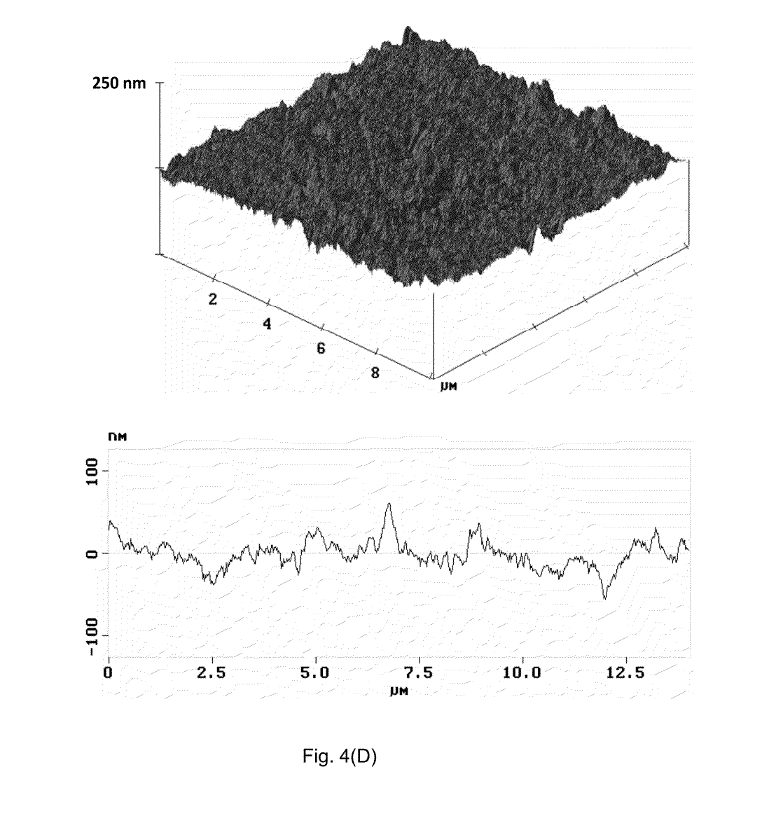

FIG. 4: AFM images and cross-section analyses of (a) a mechanically polished gold surface prior to implantation, (b) a mechanically polished gold surface after implantation, (c) a “Fenton-polished” gold surface prior to implantation and (d) a “Fenton-polished” gold surface after implantation in the peritoneal cavity of mice.

In the following, a first embodiment of a method according to the invention for improving the biocompatibility of a surface 1, a solid body surface in the schematic display of FIG. 1, will be explained with reference to the schematic display of FIG. 1. The surface 1 is brought in contact with reactive radicals 2. The radical can have a number n of unpaired electrons (indicated by a •). A radical that contains two unpaired electrons is called a diradical; in case of three unpaired electrons, it is called a triradical, etc.

The surface 1 can be the surface of a device 3, for example an implant, a sensor or a cell culture vessel, whose biocompatibility is to be improved.

The reactive radicals 2 cause the deactivation of the active centers 4 of the surface 1 that trigger the biological reactions and that have a cell- and/or tissue-damaging effect. The active center 4 is marked schematically in the Figures as a circle encompassing a star, whereby the star symbolizes the cytotoxic effect, i.e. the cell- and/or tissue-damaging property of the active center 4.

As shown on the right side in FIG. 1, the reactive radical 2 deactivates the active center 4 of the surface 1. The deactivation can for example occur through the active center 4 being split off the surface and detached from this surface as shown on the right side at the top of FIG. 1. The deactivation can also take place in that the active center 4 is transformed by the reactive radical 2 in a way that it will no longer have a cell- or tissue damaging effect, which is symbolized in a way that the star indicating the cytotoxic effect is not longer displayed at the bottom right in FIG. 1.

A further embodiment of the method according to the invention is schematically displayed in FIG. 2. In the embodiment of FIG. 2, reactive radicals 2 are created through splitting of a radical starter 5. In contrast to a reactive radical 2, the radical starter 5 is stable, i.e. less reactive and more durable. In the method of the embodiment shown in FIG. 2, the radical starter 5 is at first brought in contact with the surface 1 of the device 3. Subsequently, the radical 5 starter will be transformed into the reactive radical 2 in situ, i.e. on the spot. For the transformation, the radical starter 5 is converted into the reactive radical 2 by means of a splitting agent 6.

The splitting agent 6 can be both a chemical substance or an enzyme as well as radiation such as UV radiation, x-rays or ionizing radiation, as well as the change of a parameter, for example the temperature or the pressure, which causes splitting of the radical starter 5 into the reactive radical 2. Depending on type and texture of the surface 1, a splitting agent 6 and hence a transformation method of the radical starter 5 can be chosen, which does not modify the properties of the surface 1, except for biocompatibility, that is improved according to the invention. For example, the biocompatibility of the surface 1 can be improved without a temperature increase by means of photolysis (light irradiation) or radiolysis (ionizing radiation). This is particularly advantageous for thermosensitive surfaces.

After the radical starter 5 has been transformed into the reactive radical 2 by means of the splitting agent 6 (right side of FIG. 2), the method of the second embodiment according to the invention continues in analogy with the method shown in FIG. 1, whereby reactive radicals 2 improve the biocompatibility of the surface 1 by means of systematic deactivation of active centers 4 of the surface 1.

The theory according to the invention will be explained by means of specific experimental results in the following.

1. Reduction of the Cytotoxicity of Gold Layers

The cell activity of galvanically deposited gold layers on stainless steel wires was examined after gammasterilization. Untreated gold layers and gold layers treated with oxygen radicals were subjected to a cytotoxicity test with human adult skin fibroplasts (NHDF cells). Therefore, eluates were produced by the wires and their impact on the cell vitality of the NHDF cells was examined by means of a colorimetric assay (TTC assay) (detailed description see: N. Saucedo-Zeni et al., Int. J. Oncol. 41 (2012) 1241-1250).

The radicals were created by means of Fenton solutions and through UV photolysis of hydrogen peroxide. The following composition of the Fenton solution was used: c_{(NH}_{₄}₎_{₂}_{Fe(SO}_{₄}₎_{₂}_{.6(H}_{₂}_{O)}=0.01 mol·L⁻¹, c_{Na}_{₂}_{EDTA}=0.01 mol L⁻¹, C_{Acetate buffer}=0.1 mol·L⁻¹ and c_{H}_{₂}_{O}_{₂}=0.1 mol·L⁻¹. The overall treatment time amounted to 120 minutes, whereby the “old” Fenton solution was replaced by a fresh Fenton solution every 5 minutes.

A “705 UV digester” (Metrohm, Switzerland) was used to obtain radicals by means of UV photolysis of H₂O₂. It became apparent that a treatment of the gold layer during 30 minutes will be sufficient to completely detoxify the gold layers if a 0.3% H₂O₂ solution is used.

While the cell vitality in untreated gold layers was only between 20 and 60%, the cell vitality in gold layers after the abovementioned treatment with reactive oxygen radicals amounted to virtually 100%, i.e. the gold layers were detoxified completely through the radical treatment.

Further, the Fenton solutions used were examined for their gold content by means of ICP-AES with an “ICP-Optical Emission Spectrometer Optima 2100 DV” (PerkinElmer, USA). In the process it was found that the larger the detached quantity of gold, the higher the cell vitality (see FIG. 3).

It is known that gold ions that are released from gold implants are biologically active (A. Larsen, K. Kolind, D. S. Pedersen, P. Doering, M. Ø. Pedersen, G. Danscher, M. Penkowa, M. Stoltenberg, Histochem. Cell. Biol. 130 (2008) 681-692; G. Danscher, A. Larsen, Histochem. Cell. Biol. 133 (2010) 367-373). In case of the organisms used for the cell toxicity tests (human dermal fibroplasts), they are obviously toxic. Hence, it becomes apparent from FIG. 3 that the detached gold atoms are active centers of the surface that trigger biological reactions. The surface is detoxified through the detachment of these active centers.

2. Implantation of Gold Sheets into the Peritoneal Cavity of Mice

Six gold sheets (size: 15 mm×5 mm×0.05 mm) were at first polished mechanically with aluminum oxide powder. Three of the mechanically polished gold sheets were subsequently treated with oxygen radicals that had been created by means of Fenton solutions. Therefore, the gold sheets are dipped into a solution consisting of (NH₄)₂Fe(SO₄)₂.6(H₂O) (c_{Fe}_{²⁺}=1.10⁻³ mol L⁻¹; Merck), Na₂EDTA.2H₂O (C_{EDTA}=1.10⁻³ mol L⁻¹; Merck) and acetate buffer (c_{CH}_{₃}_{COOH}=C_{CH}_{₃}_{COO}_{^{−}}=1.10⁻² mol L⁻¹, pH=4.7; Merck) that is always produced freshly. The Fenton reaction was started by adding H₂O₂ (Merck) and the gold sheets were exposed to this solution for 5 minutes. This procedure was repeated 12 times so that the overall treatment time amounted to 120 minutes. The Fenton solution always contained c_{H}_{₂}_{O}_{₂} and c_{Fe}_{²⁺} in a 10:1 ratio.

AFM images were taken both of the mechanically polished as well as of the Fenton-treated gold sheets (see FIGS. 4a and 4c) and the roughness factors of the surfaces were determined (see Table 1). The AFM measurements were made by means of a “NanoScope I” (Digital Instruments, USA) in the contact mode.

The gold sheets were implanted into the peritoneal cavity of mice (one gold sheet per mouse). After 14 days, the gold sheets were removed from the mice and AFM images of the gold surfaces were taken (see FIGS. 4b and 4d) and the roughness factors were determined (see Table 1) once again.

Based on the AFM images and roughness factors, it becomes clear that the gold surfaces that were treated merely through mechanical polishing are straightened in the peritoneal cavity of the mice, i.e. biologically active, i.e. cell-damaging, gold is detached from the implants. The mechanically polished gold surfaces that were subsequently treated with radicals show in turn no changed roughness of the surface because the active centers are deactivated during treatment of the surface with reactive radicals. Therefore, no gold was detached from the gold surfaces in the peritoneal cavity. This proves that implants have a higher biocompatibility (are not affected) due to pre-treatment with radicals.

TABLE 1
Roughness factors of the different treated gold surfaces

| | | Roughness factor [nm] |
|---|---|---|
| Mechanically | Prior to implantation | 14.7 ± 2.1 |
| polished | After implantation | 10.2 ± 0.5 |
| “Fenton- | Prior to implantation | 12.6 ± 1.0 |
| polished” | After implantation | 13.4 ± 1.3 |

### REFERENCE SIGNS

1 Surface

2 Reactive radical

3 Device (for example implant, sensor or cell culture vessel)

4 Active center

5 Radical starter

6 Splitting agent

1. A method for improving the biocompatibility of a surface, comprising contacting the surface with at least one species of reactive radicals.

2. The method according to claim 1, wherein the reactive radicals deactivate active centers of the surface, which if active can trigger biological reactions which can result in a cell- or tissue-damaging effect.

3. The method according to claim 1, wherein the reactive radicals comprise at least an oxygen radical, a nitrogen radical, a carbon radical, a sulfur radical and/or a halogen radical.

4. The method according to claim 1, wherein the reactive radicals are created by breaking down a radical starter.

5. The method according to claim 4, wherein the radical starter is brought in contact with the surface and transformed into reactive radicals in situ.

6. The method according to claim 4, wherein the radical starter is transformed into reactive radicals by photolysis, radiolysis, thermolysis, by means of plasma, through a chemical and/or biological reaction.

7. The method according to claim 1, wherein the reactive radical is a hydroxyl radical that is formed by one of the following reactions:
a. in a Fenton reaction;
b. through photolysis of a peroxide;
c. through radiolysis of water or another oxygen compound that can be radiolyzed into hydroxyl radicals; or
d. through a plasma reaction of an oxygen compound that can be transformed into hydroxyl radicals by means of plasma treatment, preferably of water or a peroxide.

8. The method according to claim 1, wherein the surface comprises a precious metal, a precious metal compound or alloy, or a polymer.

9. The method according to claim 1, wherein the surface is the surface of an implant, a sensor or a cell culture vessel.

10. A device comprising a biocompatible surface treated according to the method of claim 1.

11. The method according to claim 1, wherein the reactive radicals comprise an oxygen radical.

12. The method according to claim 1, wherein the reactive radicals comprise one or more oxygen radicals selected from one or more of hyperoxide anions, hydroxyl radicals, hydroperoxyl radicals, peroxyl radicals, and/or alcoxyl radicals.

13. The method according to claim 1, wherein the surface comprises gold.

14. A device comprising a biocompatible surface treated according to the method of claim 1, wherein the surface comprises a precious metal, a precious metal compound or alloy, or a polymer.

15. The device according to claim 1, wherein the device is an implant, sensor or cell culture vessel.

16. The device according to claim 1, wherein the surface comprises gold.

17. A device comprising a biocompatible surface treated according to the method of claim 11.

18. The device according to claim 17, wherein the surface comprises a precious metal, a precious metal compound or alloy, or a polymer.

19. The device according to claim 17, wherein the surface comprises gold.
